(19) United States
(12) Patent Application Publication
(10) Pub. No.: US 2004/0198609 A1
(43) Pub. Date: Oct. 7, 2004

(54) 4-alkyl-substituted thienyloxy-pyridines

(76) Inventors: Michael Hoffmann, Ludwigshafen (DE); Liliana Parra Rapado, Mannheim (DE); Wolfgang von Deyn, Neustadt (DE); Ernst Baumann, Dudenhofen (DE); Markus Kordes, Frankenthal (DE); Ulf Misslitz, Neustadt (DE); Matthias Witschel, Bad Durkheim (DE); Cyrill Zagar, Mannheim (DE); Andreas Landes, Romerberg Heiligenstein (DE)

Correspondence Address: KEIL & WEINKAUF, 1350 CONNECTICUT AVENUE, N.W., WASHINGTON, DC, 20036, US

(21) Appl. No.: 10/487,549

(22) PCT Filed: Aug. 31, 2002
(86) PCT No.: PCT/EP02/09751

(30) Foreign Application Priority Data
Sep. 7, 2001 (DE) 101 44 073.1

(51) Int. Cl. A01N 43/48; C 07D 4 9/14

(52) U.S. Cl. 504/253; 546/275.4

(57) ABSTRACT

4-Alkyl-substituted thienyloxypyridines of the formula I

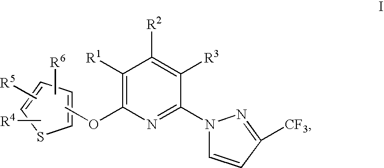

where:

R¹, R³ are hydrogen, halogen, cyano, nitro, alkyl, haloalkyl, alkoxy or haloalkoxy;

R² is alkyl or cycloalkyl;

R⁴, R⁵, R⁶ are hydrogen, halogen, cyano, alkyl, haloalkyl, alkoxy, haloalkoxy, alkylthio, haloalkylthio, alkylsulfonyl or haloalkylsulfonyl;

where R² is not methyl if R¹ and R³ are hydrogen;

and their agriculturally useful salts;

processes and intermediates for their preparation, and the use of these compounds or of the compositions comprising them for controlling undesirable plants are described.

The present invention relates to 4-alkyl-substituted thienyloxypyridines of the formula I

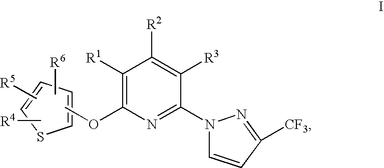

where

R¹, R³ are hydrogen, halogen, cyano, nitro, C₁-C₆-alkyl, C₁-C₆-haloalkyl, C₁-C₆-alkoxy or C₁-C₆-haloalkoxy;

R² is C₁-C₆-alkyl or C₃-C₆-cycloalkyl;

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or C₁-C₆-haloalkylsulfonyl;

where R² is not methyl if R¹ and R³ are hydrogen;

and their agriculturally useful salts.

Moreover, the invention relates to intermediates and processes for preparing compounds of the formula I, to compositions comprising them and to the use of these derivatives or of the compositions comprising these derivatives for controlling harmful plants. WO 99/24427 and EP-A-1 101 764 disclose herbidically active thienyloxyazines and 2-aryloxy-6-pyrazole pyridines.

However, the herbicidal properties of the prior-art compounds and/or their compatibility with crop plants are not entirely satisfactory.

It is an object of the present invention to provide in particular herbicidally active compounds having improved properties.

We have found that this object is achieved by the 4-alkyl-substituted thienyloxypyridines of the formula I and their herbidical action.

Furthermore, we have found herbicidal compositions which comprise the compounds I and have very good herbicidal action. Moreover, we have found processes for preparing these compositions and methods for controlling undesirable vegetation using the compounds I.

Depending on the substitition pattern, the compounds of the formula I may contain one or more centers of chirality, in which case they are present as enantiomers or mixtures of diastereomers. The invention provides both the pure enantiomers or diastereomers and their mixtures.

The compounds of the formula I can also be present in the form of their agriculturally useful salts, the type of salt generally being immaterial. Suitable are, in general, the salts of those cations and the acid addition salts of those acids whose cations and anions, respectively, do not adversely affect the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, 1 to 4 hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri (C₁-C₄-alkyl) sulfoxonium.

Anions of useful acid addition salts are preferably chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and also the anions of C₁-C₄-alkanoic acids, preferably formate, acetate, propionate and butyrate.

The organic moieties mentioned for the substituents R¹-R⁶ are collective terms for individual enumerations of the individual group members. All hydrocarbon chains, i.e. all alkyl, haloalkyl, alkoxy, haloalkoxy, alkylthio, haloalkylthio, alkylsulfonyl and haloalkylsulfonyl moieties can be straight-chain or branched. Unless indicated otherwise, halogenated substituents preferably carry one to five, in particular one to three, identical or different halogen atoms. The term ‘halogen’ denotes in each case fluorine, chlorine, bromine or iodine.

Examples of other meanings are:

C₂-C₄-alkyl: for example ethyl, propyl, 1-methylethyl, butyl, 1-methylpropyl, 2-methylpropyl and 1,1-dimethylethyl;

C₁-C₄-alkyl: and the alkyl moieties of hydroxy-C₁-C₄-alkyl, hydroxy-C₁-C₄-alkoxy-C₁-C₄-alkyl, tri(C₁-C₄-alkyl)sulfonium and tri(C₁-C₄-alkyl)sulfoxonium: C₂-C₄-alkyl as mentioned above and, for example, methyl;

C₂-C₆-alkyl: C₂-C₄-alkyl as mentioned above, and also, for example, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;

C₁-C₆-alkyl: C₂-C₆-alkyl as mentioned above and, for example, methyl;

C₁-C₆-haloalkyl: a C₁-C₆-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;

C₁-C₄-alkoxy and the alkoxy moieties of C₁-C₄-alkoxy-C₁-C₄-alkyl, hydroxy-C₁-C₄-alkoxy-C₁-C₄-alkyl: for example methoxy, ethoxy, propoxy, 1-methylethoxy, butoxy, 1-methylpropoxy, 2-methylpropoxy and 1,1-dimethylethoxy;

C₁-C₆-alkoxy: C₁-C₄-alkoxy as mentioned above and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

C₁-C₆-haloalkoxy: a C₁-C₆-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloro-propoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluoro-butoxy, 4-chlorobutoxy, 4-bromobutoxy, nonafluorobutoxy, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;

C₁-C₆-alkylthio: for example methylthio, ethylthio, propylthio, 1-methylethylthio, butylthio, 1-methylpropylthio, 2-methylpropylthio and 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

C₁-C₆-haloalkylthio: a C₁-C₆-alkylthio radical as mentioned above which is partially or fully substituted by fluorine, chlorine, bromine and/or iodine, i.e., for example, fluoromethylthio, difluoromethylthio, trifluoromethylthio, chlorodifluoromethylthio, bromodifluoromethylthio, 2-fluoroethylthio, 2-chloroethylthio, 2-bromoethylthio, 2-iodoethylthio, 2,2-difluoroethylthio, 2,2,2-trifluoroethylthio, 2,2,2-trichloroethylthio, 2-chloro-2-fluoroethylthio, 2-chloro-2,2-difluoroethylthio, 2,2-dichloro-2-fluoroethylthio, pentafluoroethylthio, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2,3-dichloropropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio., 4-chlorobutylthio, 4-bromobutylthio, nonafluorobutylthio, 5-fluoropentylthio, 5-chloropentylthio, 5-bromopentylthio, 5-iodopentylthio, undecafluoropentylthio, 6-fluorohexylthio, 6-chlorohexylthio, 6-bromohexylthio, 6-iodohexylthio and dodecafluorohexylthio;

C₁-C₆-alkylsulfonyl (C₁-C₆-alkyl-S(═O) ₂—): for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;

C₁-C₆-haloalkylsulfonyl: a C₁-C₆-alkylsulfonyl radical as mentioned above which is partially or fully substituted by fluorine, chlorine, bromine and/or iodine, i.e., for example, fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6-fluorohexylsulfonyl, 6-bromohexylsulfonyl, 6-iodohexylsulfonyl and dodecafluorohexylsulfonyl;

C₃-C₆-cycloalkyl: for example, cyclopropyl, cyclobutyl, cyclopentyl and cyclohexyl.

In a particular embodiment, the variables of the compounds of the formula I have the following meanings, these meanings, both on their own and in combination with one another, being particular embodiments of the compounds of the formula I:

Preference is given to the 4-alkyl-substituted thienyloxypyridines of the formula I in which

R¹, R³ are hydrogen, halogen, cyano, C₁-C₆-alkyl or C₁-C₆-haloalkyl;

particularly preferably hydrogen, halogen, such as fluorine, chlorine or bromine, or C₁-C₆-alkyl, such as methyl or ethyl, or C₁-C₆-haloalkyl such as trifluoromethyl;

with particular preference hydrogen, fluorine, chlorine or methyl.

Moreover, preference is given to the 4-alkyl-substituted thienyloxypyridines of the formula I in which

R¹ is hydrogen; and

R³ is hydrogen, halogen, cyano, C₁-C₆-alkyl or C₁-C₆-haloalkyl;

particularly preferably hydrogen, halogen, such as fluorine, chlorine or bromine, or C₁-C₆-alkyl, such as methyl or ethyl, or C₁-C₆-haloalkyl such as trifluoromethyl;

with particular preference hydrogen, fluorine, chlorine or methyl.

Moreover, preference is given to the 4-alkyl-substituted thienyloxypyridines of the formula I in which

R¹ is hydrogen, halogen, cyano, C₁-C₆-alkyl or C₁-C₆-haloalkyl;

particularly preferably hydrogen, halogen, such as fluorine, chlorine or bromine, or C₁-C₆-alkyl, such as methyl or ethyl, or C₁-C₆-haloalkyl such as trifluoromethyl;

with particular preference hydrogen, fluorine, chlorine or methyl; and

R³ is hydrogen.

Preference is also given to the 4-alkyl-substituted thienyloxypyridines of the formula I in which

R² is methyl,

if R¹ is not hydrogen.

Preference is also given to the 4-alkyl-substituted thienyloxypyridines of the formula I in which

R² is C₂-C₆-alkyl;

particularly preferably C₂-C₄-alkyl, such as ethyl or isopropyl;

with particular preference ethyl or isopropyl.

Preference is also given to the 4-alkyl-substituted thienyloxypyridines of the formula I in which

R² is C₃-C₆-cycloalkyl;

particularly preferably cyclopropyl;

Moreover, preference is given to the 4-alkyl-substituted thienyloxypyridines of the formula I in which

R¹ is hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl;

R² is C₁-C₆-alkyl;

particularly preferably C₁-C₄-alkyl, such as methyl, ethyl or isopropyl;

with particular preference methyl or ethyl; and

R³ is halogen, cyano, C₁-C₆-alkyl or C₁-C₆-haloalkyl;

particularly preferably halogen, such as fluorine, chlorine or bromine, C₁-C₆-alkyl, such as methyl or ethyl;

with particular preference fluorine, chlorine or methyl.

Preference is furthermore given to the 4-alkyl-substituted thienyloxypyridines of the formula I in which

R¹, R³ are hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl; and

R² is C₂-C₆-alkyl;

particularly preferably C₂-C₄-alkyl, such as ethyl or isopropyl;

with particular preference ethyl or isopropyl.

In addition, preference is given to the 4-alkyl-substituted thienyloxypyridines of the formula I in which

R¹ is hydrogen;

R² is C₂-C₆-alkyl;

particularly preferably C₂-C₄-alkyl, such as ethyl or isopropyl;

with particular preference ethyl or isopropyl;

R³ is hydrogen.

Likewise, preference is given to the 4-alkyl-substituted thienyloxypyridines of the formula I in which

R¹, R³ are halogen, cyano, C₁-C₆-alkyl or C₁-C₆-haloalkyl;

particularly preferably halogen, such as fluorine, chlorine or bromine,

C₁-C₆-alkyl, such as methyl or ethyl;

with particular preference fluorine, chlorine or methyl; and

R² is methyl.

Likwise, preference is given to the 4-alkyl-substituted thienyloxypyridines of the formula I in which

R¹ is hydrogen;

R² is methyl; and

R³ is halogen, cyano, C₁-C₆-alkyl or C₁-C₆-haloalkyl;

particularly preferably halogen, such as fluorine, chlorine or bromine, C₁-C₆-alkyl, such as methyl or ethyl;

with particular preference fluorine, chlorine or methyl;

Preference is also given to the 4-alkyl-substituted thienyloxypyridines of the formula I in which

R¹ is halogen, cyano, C₁-C₆-alkyl or C₁-C₆-haloalkyl;

particularly preferably halogen, such as fluorine, chlorine or bromine, C₁-C₆-alkyl, such as methyl or ethyl;

with particular preference fluorine, chlorine or methyl;

R² is methyl; and

R³ is hydrogen

Preference is also given to 4-alkyl-substituted thienyloxypyridines of the formula I in which in each case independently of one another

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl, C₁-C₆-haloalkylsulfonyl;

particularly preferably hydrogen, halogen, cyano, C₁-C₆-alkyl, C₁-C₆-haloalkyl, C₁-C₆-haloalkoxy, C₁-C₆-alkylsulfonyl or C₁-C₆-haloalkylsulfonyl;

with particular preference hydrogen, halogen, such as fluorine, chlorine or bromine, cyano, C₁-C₆-haloalkyl, such as trifluor Qmethyl, trichloromethyl or dichloromethyl, or C₁-C₆-haloalkoxy, such as difluoromethoxy or trifluoromethoxy;

very preferably hydrogen, fluorine, chlorine, trifluoromethyl or difluoromethoxy;

Preference is also given to the 4-alkyl-substituted thienyloxypyridines of the formula I in which R⁶ is hydrogen and in each case independently of one another

R⁴, R⁵ are hydrogen, halogen, C₁-C₆-alkyl or C₁-C₆-haloalkyl;

particularly preferably hydrogen, chlorine, methyl or trifluoromethyl.

Particular preference is also given to the 4-alkyl-substituted thienyloxypyridines of the formula I in which the thienyl radical is attached in the 3-position via the oxygen atom to the pyridine skeleton and substituted by R⁴ and R⁵ in the 4- and 5-positions, respectively.

Particular preference is also given to the 4-alkyl-substituted thienyloxypyridines of the formula I in which the thienyl radical is attached in the 2- and 5-position via the oxygen atom to the pyridine skeleton and substituted by R⁴ and R⁵ in the 4- and 5-positions, respectively.

Preference is also given to the 4-alkyl-substituted thienyloxypyridines of the formula I in which R⁵ and R⁶ are hydrogen and

R⁴ is halogen, cyano, C₁-C₆-alkyl, C₁-C₆-haloalkyl or C₁-C₆-haloalkoxy;

particularly preferably halogen or C₁-C₆-haloalkyl;

very preferably fluorine, chlorine or trifluoromethyl.

Particular preference is also given to the 4-alkyl-substituted thienyloxypyridines of the formula I in which the thienyl radical is attached in the 3-position via the oxygen atom to the pyridine skeleton and substituted by R⁴ in the 5-position.

Particular preference is also given to the 4-alkyl-substituted thienyloxypyridines of the formula I in which the thienyl radical is attached in the 2-position via the oxygen atom to the pyridine skeleton and substituted by R⁴ in the 5-position.

Most preference is given to compounds of the formula Ia (where R⁴=5-CF₃, R⁵═H, R⁶═H; the thienyl radical is attached in the 3-position via an oxygen atom to the pyridine skeleton), in particular to the compounds Ia.1 to Ia.26 of Table 1, where the definitions of the variables R¹ to R⁶ play a particular role for the compounds according to the invention, not only in combination with one another but in each case also on their own.

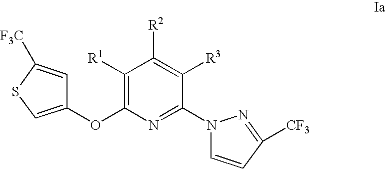

TABLE 1

| No. | R¹ | R² | R³ |
|---|---|---|---|
| Ia.1 | CH₃ | CH₃ | CH₃ |
| Ia.2 | F | CH₃ | F |
| Ia.3 | Cl | CH₃ | Cl |
| Ia.4 | Br | CH₃ | Br |
| Ia.5 | OCH₃ | CH₃ | OCH₃ |
| Ia.6 | CN | CH₃ | CN |
| Ia.7 | CN | CH₃ | H |
| Ia.8 | H | CH₃ | CN |
| Ia.9 | NO₂ | CH₃ | H |
| Ia.10 | H | CH₃ | NO₂ |
| Ia.11 | Cl | CH₃ | H |
| Ia.12 | H | CH₃ | Cl |
| Ia.13 | CN | CH₃ | Cl |
| Ia.14 | Cl | CH₃ | CN |
| Ia.15 | CF₃ | CH₃ | Cl |
| Ia.16 | Cl | CH₃ | CF₃ |
| Ia.17 | CN | CH₂CH₃ | CN |
| Ia.18 | H | CH₂CH₃ | H |
| Ia.19 | F | CH₂CH₃ | F |
| Ia.20 | Cl | CH₂CH₃ | Cl |
| Ia.21 | H | (CH₂)₂CH₃ | H |
| Ia.22 | F | (CH₂)₂CH₃ | F |
| Ia.23 | Cl | (CH₂)₂CH₃ | Cl |
| Ia.24 | H | CH(CH₃)₃ | H |
| Ia.25 | F | CH(CH₃)₂ | F |
| Ia.26 | Cl | CH(CH₃)₂ | Cl |

Most preference is also given to the compounds of the formula Ib, in particular to the compounds Ib.1 to Ib.26 which differ from the corresponding compounds Ia.1 to Ia.26 in that R⁴ in the 5-position is chlorine.

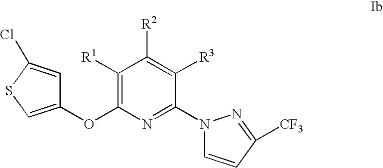

Most preference is also given to the compounds of the formula Ic, in particular to the compounds Ic.1 to Ic.26 which differ from the corresponding compounds Ia.1 to Ia.26 in that the thienyl radical is attached in the 2-position via the oxygen atom to the pyridine skeleton.

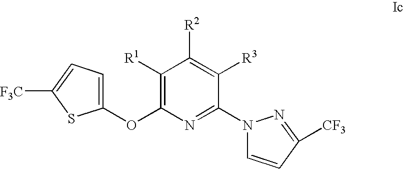

Most preference is also given to the compounds of the formula Id, in particular to the compounds Id.1 to Id.26 which differ from the corresponding compounds Ia.1 to Ia.26 in that R⁴ in the 5position is chlorine and the thienyl radical is attached in the 2-position via the oxygen atom to the pyridine skeleton.

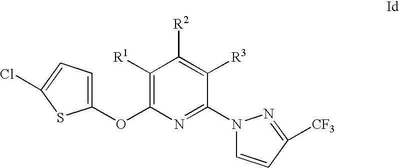

Most preference is also given to the compounds of the formula Ie, in particular to the compounds Ie.1 to Ie.26 which differ from the corresponding compounds Ia.1 to Ia.26 in that R⁴ in the 4-position is trifluoromethyl and the thienyl radical is attached in the 2-position via the oxygen atom to the pyridine skeleton.

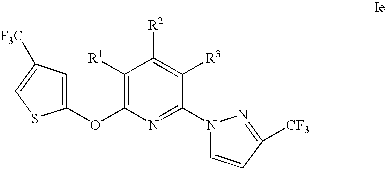

Most preference is also given to the compounds of the formula If, in particular to the compounds If.1 to If.26 which differ from the corresponding compounds Ia.1 to Ia.26 in that R⁴ in the 4-position is chlorine and the thienyl radical is attached in the 2-position via the oxygen atom to the pyridine skeleton.

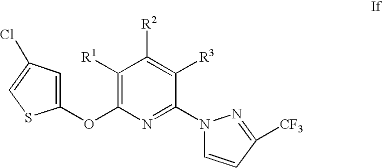

The 4-alkyl-substituted thienyloxypyridines of the formula I can be obtained by various methods, for example by the processes below.

Process A

The 3-trifluoromethyl-1H-pyrazol-1-yl-substituted pyridines of the formula III are obtained from pyridines of the formula V by reaction with 3-trifluoromethyl-1H-pyrazole IV. L¹ and L² are nucleophilically displaceable leaving groups, such as halogen, for example fluorine, chlorine and bromine, C₁-C₄-alkylsulfonyl, such as, for example, methylsulfonyl, C₁-C₄-alkylsulfonyloxy, such as, for example, methylsulfonyloxy, C₁-C₄-haloalkylsulfonyloxy or trialkylammonium, preferably fluorine, chlorine or bromine, C₁-C₄-alkylsulfonyl, such as, for example, methylsulfonyl, or C₁-C₄-haloalkylsulfonyloxy, such as, for example, trifluoro-ethylsulfonyloxy. These compounds are then reacted with hydroxythiophenes of the formula II to give 4-alkyl-substituted thienyloxypyridines of the formula I:

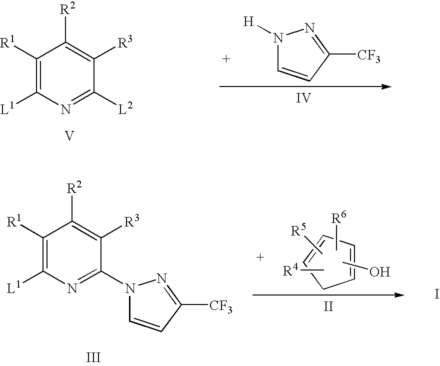

The conversion of pyridines of the formula V into 3-trifluoromethyl-1H-pyrazol-1-yl-substituted pyridines of the formula III is usually carried out at 0°-200°, preferably at 10° C.-100°, in an inert organic solvent in the presence of a base [cf. WO 98/40379; EP 1 101 764].

Suitable solvents are aliphatic hydrocarbons, such as pentane, hexane, cyclohexane and mixtures of C₅-C₈-alkanes, ethers, such as diethyl ether, diisopropyl ether, tert-butyl methyl ether, dioxane, anisole and tetrahydrofuran; nitriles, such as acetonitrile and propionitrile, and also dimethyl sulfoxide, dimethylformamide and dimethylacetamide, particularly preferably acetonitrile and dimethylformamide.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal amides, such as-lithium amide, sodium amide and potassium amide, alkali metal and alkaline earth metal carbonates, such as lithium carbonate, potassium carbonate and calcium carbonate, and also alkali metal and alkaline earth metal alkoxides, such as sodium methoxide, sodium ethoxide, potassium ethoxide, potassium tert-butoxide and potassium tert-pentoxide; organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to potassium carbonate, sodium hydride, potassium tert-butoxide and potassium tert-pentoxide.

The bases are generally employed in equimolar amounts; however, it is also possible to employ them in excess or, if appropriate, as solvent.

The starting materials are generally reacted with one another in equimolar amounts. In terms of yield, it may be advantageous to employ an excess of V, based on IV.

The conversion of 3-trifluoromethyl-1H-pyrazol-1-yl-substituted pyridines of the formula III into 4-alkyl-substituted thiophenyloxypyridines of the formula I is usually carried out at 50° C.-200° C., preferably at 50° C.-150° C., in an inert organic solvent in the presence of a base [cf. WO 98/40379; EP 1 101 764].

Suitable solvents are aliphatic hydrocarbons, such as pentane, hexane, cyclohexane and mixtures of C₅-C₈-alkanes, ethers, such as diethyl ether, diisopropyl ether, tert-butyl methyl ether, dioxane, anisole, tetrahydrofuran and diethylene glycol dimethyl ether, nitriles, such as acetonitrile and propionitrile, and also dimethyl sulfoxide, dimethylformamide, dimethylacetamide, N-methylpyrrolidone and sulfolane, particularly preferably acetonitrile, diethylene glycol dimethyl ether, dimethylformamide, N-methylpyrrolidone and sulfolane.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal and alkaline earth metal alkoxides, such as sodium methoxide, sodium ethoxide, potassium ethoxide, potassium tert-butoxide and potassium tert-pentoxide; organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to potassium carbonate, sodium hydride, potassium tert-butoxide and potassium tert-pentoxide.

The bases are generally employed in equimolar amounts; however, they can also be used in excess or, if appropriate, as solvent.

The starting materials are generally reacted with one another in equimolar amounts. In terms of yield, it may be advantageous to employ an excess of II, based on III.

The starting materials required for preparing the compounds I are known from the literature or can be prepared in accordance with the literature cited [cf. EP 1 101 764].

The reaction mixtures are worked up in a customary manner, for example by mixing with water, separating the phases and, if appropriate, purifying the crude products by chromatography. Some of the intermediates and end products are obtained in the form of colorless or slightly brownish, viscous oils which, under reduced pressure and at moderately elevated temperature, can be freed from volatile fractions or purified. If the intermediates and end products are obtained as solids, purification can also be effected by recrystallization or digestion.

Process B

A dihalopyridine of the formula V (where L¹=Hal and L²=Hal′) is reacted with sodium mercaptan or potassium mercaptan of the formula VIII to give pyridines of the formula VII. Here, Ra is C₁-C₆-alkyl, preferably methyl. The pyridines of the formula VII can then be reacted with a pyrazole of the formula IV to give 4-alkyl-substituted pyridines of the formula VI:

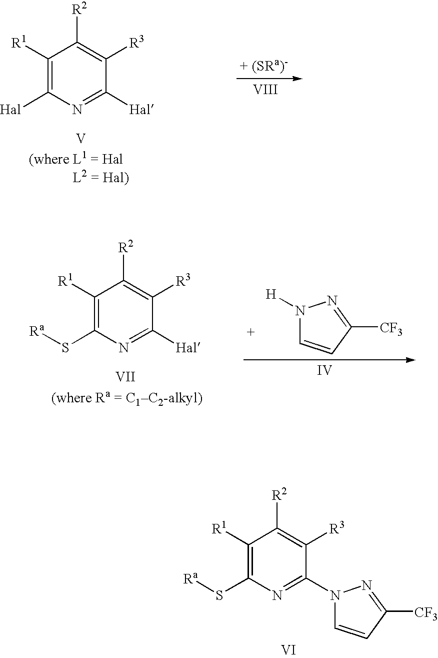

The conversion into pyridines of the formula VII is usually carried out at 0° C.-80° C. in an inert organic solvent [cf. WO 98/40379].

Suitable solvents are ethers, such as diethyl ether, diisopropyl ether, tert-butyl methyl ether, dioxane, anisole and tetrahydrofuran, particularly preferably tetrahydrofuran. It is also possible to use mixtures of the solvents mentioned.

The starting materials are generally reacted with one another in equimolar amounts.

Work-up can be carried out in a manner known per se to afford the product.

The conversion of pyridines of the formula VII into 4-alkyl-substituted pyridines of the formula VI is usually carried out at 50° C.-200° C., preferably at 50° C.-150° C., analogously to the conversion of V into III (cf. process A).

The 4-alkyl-substituted pyridines of the formula VI are then oxidized to give compounds of the formula III (where L¹=SO₂R^{a}). By further reaction with hydroxythiophenes of the formula II, the 4-alkyl-substituted thienyloxypyridines of the formula I are obtained:

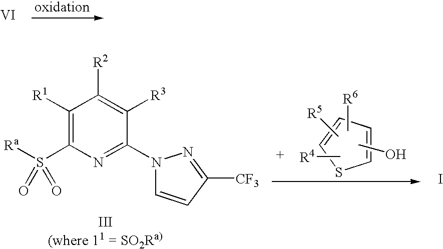

The oxidation is usually carried out at 0° C.-100° C., preferably at 25° C., in an inert organic solvent [cf. J. March, Organic Chemistry, 1992, 1201-1203].

Suitable oxidizing agents are, for example, metachloroperbenzoic acid, peroxyacetic acid, trifluoroperoxyacetic acid, hydrogen peroxide, sodium periodate or Oxone®. It may be advantageous to carry out the reaction in the presence of a catalyst, for example sodium tungstate.

Suitable solvents are halogenated hydrocarbons, such as methylene chloride, chloroform and chlorobenzene, alcohols, such as methanol, ethanol, n-propanol, isopropanol, n-butanol and tert-butanol.

The starting materials are generally reacted with one another in equimolar amounts. In terms of yield, it may be advantageous to employ an excess of oxidizing agent, based on VI.

Work-up can be carried out in a manner known per se to afford the product.

The reaction of compounds of the formula III with hydroxythiophenes of the formula II is carried out under the same conditions as the conversion of III into I (cf. process A).

Process C

It is also possible to synthesize the nitrogen heterocycle directly from a corresponding aminopyridine. This gives 4-alkyl-substituted pyridines which can then be modified further by the reactions shown above. By way of example, this may be demonstrated using the conversion of the aminopyridines of the formula IX into the 3-trifluoromethyl-1H-pyrazol-1-yl-substituted pyridines of the formula III (where L¹=chlorine). However, the heterocycle can also be constructed at a different stage of the variants A, B and D to F shown.

The aminopyridine of the formula IX is initially converted into the diazonium compound, giving, after hydrogenation, the corresponding pyridinehydrazine derivative. This is then reacted with 1,3-dicarbonyl compounds, enol esters or 1-alkynyl ketones in a cyclocondensation to give the desired pyrazole:

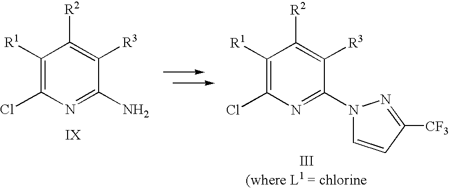

The resulting 3-trifluoromethyl-1H-pyrazol-1-yl-substituted pyridines of the formula III can then be modified further by the reactions presented here.

The abovementioned reactions are generally known from the literature and described, inter alia, in T. Eicher, S. Hauptmann, Chemie der Heterocyclen [Chemistry of heterocycles], 1994, 183; A. S. Tomcufcik, L. N. Starker, The Chemistry of Heterocyclic Compounds, Pyridine and its Derivatives part 3, 1962, 34-35.

Process D

In this variant, pyridines of the formula XII are initially reacted with a pyrazole of the formula IV under the same reaction conditions which can also be used to convert V into III (cf. process A). The product is then oxidized giving a pyridine N-oxide of the formula X and, after halogenation, a 3-trifluoromethyl-1H-pyrazol-1-yl-substituted pyridine of the formula III where L¹=Hal is obtained. 4-Alkyl-substituted thienyloxypyridines of the formula I are obtained by analogous reaction of the 3-trifluoromethyl-1H-pyrazol-1-yl-substituted pyridines of the formula III with hydroxythiophenes of the formula II, as described in process A.

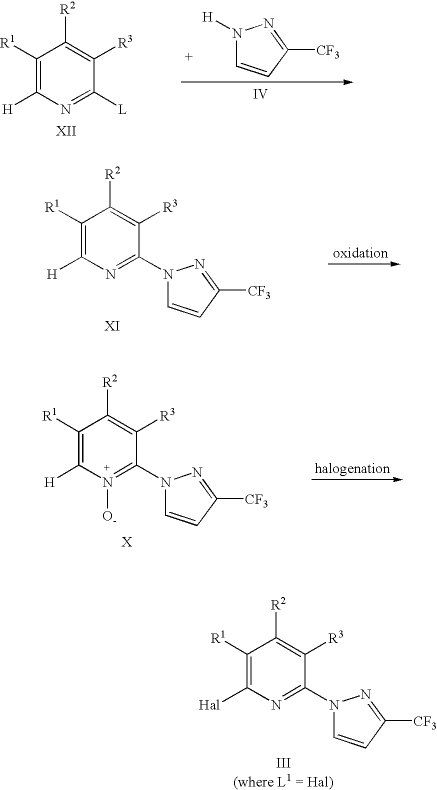

The oxidation of the pyridines of the formula XI to give pyridine N-oxides of the formula X is usually carried out at 0° C.-100° C., preferably at 0° C.-25° C., in an inert organic solvent [cf. G. C. Finger et al., J. Am. Chem. Soc. 81 (1959), 2674-2675; M. Tiecco et al., Tetrahedron 42 (1986), 1475-1485].

Suitable oxidizing agents are, for example, metachloroperbenzoic acid, peroxyacetic acid or hydrogen peroxide.

Suitable solvents are halogenated hydrocarbons, such as methylene chloride, chloroform and chlorobenzene, and alcohols, such as methanol, ethanol, n-propanol, isopropanol, n-butanol and tert-butanol.

The starting materials are generally reacted with one another in equimolar amounts. In terms of yield, it may be advantageous to employ an excess of oxidizing agent, based on XI.

Work-up can be carried out in a manner known per se to afford the product.

The halogenation of the pyridine N-oxides of the formula X to give 3-trifluoromethyl-1H-pyrazol-1-yl-substituted pyridines of the formula III where L¹=Hal is usually carried out at 25° C.-200° C., preferably at 80° C.-150° C., in an inert organic solvent [cf. H. E. Mertel, The Chemistry of Heterocyclic Compounds, Pyridine and its Derivatives part 2, 1961, 305-307].

Suitable halogenating agents are, for example, phosphorus oxytrichloride, phosphorus oxytribromide or sulfuryl chloride.

A halogenating agent which is also suitable is thionyl chloride.

Suitable solvents are aromatic hydrocarbons, such as toluene and o-, m- and p-xylene.

The starting materials are generally reacted with one another in equimolar amounts. In terms of yield, it may be advantageous to employ an excess of halogenating agent, based on X.

Work-up can be carried out in a manner known per se to afford the product.

Process E

Thienyloxypyridines of the formula XIII are obtained by reacting pyridines of the formula V with hydroxythiophenes of the formula II (cf. EP.955 300). This reaction is usually carried out at 25° C.-200° C., preferably at 80° C.-150° C., analogously to the reaction conditions described for the conversion of III into I (cf. process A). The thienyloxypyridines of the formula XIII are then reacted, analogously to the conversion of V into III (cf. process A), with pyrazole derivatives of the formula IV (cf. EP 1 101 764):

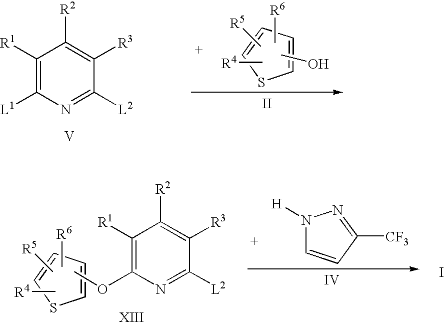

Alternatively, the conversion of XIII into I can also be carried out catalytically using nickel or palladium. In this case, the reaction is usually carried out at 25° C.-130° C. in an inert organic solvent in the presence of a base [cf. B. Gradel et al., Tetrahedron Lett. 42 (2001), 5689-5692; J. F. Hartwig et al., J. Am. Chem. Soc. 120 (1998), 827-828].

Here, L² is usually a halogen atom, such as, for example, chlorine, bromine or iodine, or another leaving group, such as, for example, trifluoromethylsulfonyloxy.

Suitable catalysts are, for example, nickel or palladium ligand complexes in which the metal is present in oxidation stage 0, preferably nickel(II) or palladium(II) salts. The reaction with nickel(II) or palladium(II) salts is preferably carried out in the presence of complex ligands.

Suitable nickel(0) complexes are, for example, nickel carbene complexes.

Suitable palladium(0) complex ligands are, for example, tetrakis(triphenylphosphine)palladium, palladium(diphenyl-phosphineferrocene) dichloride {[PdCl₂(dppf)]} or tris-(dibenzylideneacetone)dipalladium Pd₂(dba) 3.

Suitable nickel(II) salts are, for example, nickel acetate and nickel acetylacetonate.

Suitable palladium(II) salts are, for example, palladium acetate and palladium chloride. The reaction is preferably carried out in the presence of complex ligands, such as, for example, diphenylphosphineferrocene (dppf).

The complex nickel salts can be prepared in a manner known per se from commercially available nickel salts, such as nickel chloride or nickel acetate, and the corresponding phosphines, such as, for example, triphenylphosphine or 1,2-bis(triphenylphosphino)ethane, or commercially available imidazolinium salts. Many complex nickel salts are also commercially available.

The complex palladium salts can be prepared in a manner known per se from commercially available palladium salts, such as palladium chloride or palladium acetate, and the corresponding phosphines, such as, for example, triphenylphosphine or 1,2-bis(diphenylphosphino)ethane. Many complex palladium salts are also commercially available. Preferred palladium salts are [(R)-(+)-2,2′-bis(diphenylphosphino)-1,1′-binaphthyl]-palladium(II) chloride, bis(triphenyl-phosphine)palladium(II) acetate and, in particular, bis(triphenylphosphine)palladium(II) chloride.

The catalyst is generally employed in a concentration of from 0.05 to 5 mol %, preferably from 1 to 3 mol %.

Suitable solvents are aromatic hydrocarbons, such as toluene, o-, m- and p-xylene, ethers, such as diethyl ether, diisopropyl ether, tert-butyl methyl ether, dioxane, anisole and tetrahydrofuran, and also dimethylformamide.

Suitable bases are, in general, inorganic compounds, such as alkali metal and alkaline earth metal hydroxides such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal and alkaline earth metal carbonates, such as sodium carbonate, potassium carbonate and cesium carbonate, and also alkali metal and alkaline earth metal alkoxides, such as sodium methoxide, sodium ethoxide, potassium ethoxide and potassium tert-butoxide.

The bases are generally employed in equimolar amounts.

The starting materials are generally reacted with one another in equimolar amounts. In terms of yield, it may be advantageous to employ an excess of IV, based on XIII.

Work-up can be carried out in a manner known per se to afford the product.

Process F

Alternatively to process E, dithienyloxy-substituted pyridines of the formula XIV are obtained by reacting pyridines of the formula V with an excess of a hydroxythiophene of the formula II (cf. EP 955 300). The reaction is preferably carried out using a double-equimolar ratio of II to V. This reaction is carried out analogously to the reaction conditions described for the conversion of III into I (cf. process A). The dithienyloxy-substituted pyridines of the formula XIV are then, usually at 25° C.-200° C., preferably at 80° C.-150° C., reacted analogously to the conversion of V into III (cf. process A) with pyrazoles of the formula IV (cf. EP 1 101 764):

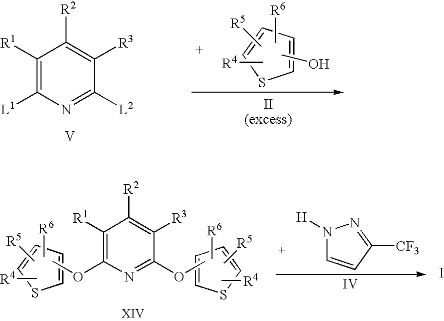

3-Trifluoromethyl-1H-pyrazol-1-yl-substituted pyridine derivatives of the formula III

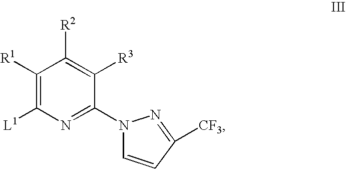

where R¹, R² and R³ are as defined for compounds of the formula I and L¹ is a nucleophilically displaceable leaving group, such as halogen, for example chlorine, bromine or iodine, C₁-C₄-alkylsulfonyl, C₁-C₄-alkylsulfonyloxy, C₁-C₄-haloalkylsulfonyloxy or trialkylammonium, preferably fluorine, chlorine or bromine, C₁-C₄-alkylsulfonyl, such as, for example, methylsulfonyl, or C₁-C₄-haloalkylsulfonyloxy, such as, for example, trifluoromethylsulfonyloxy, also form part of the subject matter of this invention.

The particularly preferred embodiments of the intermediates with respect to the variables correspond to those of the radicals R¹, R² and R³ of the formula I.

Particular preference is given to compounds of the formula III in which

R¹, R³ are hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl; and

R² is C₁-C₆-alkyl;

particularly preferably C₁-C₄-alkyl, such as methyl, ethyl or isopropyl;

with particular preference methyl or ethyl;

where R² is not methyl if R¹ and R³ are hydrogen.

Specially preferred are compounds of the formula III in which

R¹ is hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl;

R² is C₁-C₆-alkyl;

particularly preferably C₁-C₄-alkyl, such as methyl, ethyl or isopropyl;

with particular preference methyl or ethyl; and

R³ is halogen, cyano, C₁-C₆-alkyl or C₁-C₆-haloalkyl;

particularly preferably halogen, such as fluorine, chlorine or bromine, C₁-C₆-alkyl, such as methyl or ethyl;

with particular preference fluorine, chlorine or methyl.

Thienyloxypyridine derivatives of the formula XIII

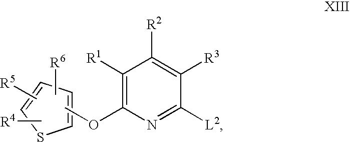

where R₁, R², R³, R⁴, R⁵ and R⁶ are as defined for compounds of the formula I and L² is a nucleophilically displaceable leaving group, such as halogen, for example fluorine, chlorine or bromine, C₁-C₄-alkylsulfonyl, C₁-C₄-alkylsulfonyloxy, such as, for example, methylsulfonyloxy, C₁-C₄-haloalkylsulfonyloxy or trialkylammonium, preferably chlorine and bromine, C₁-C₄-alkylsulfonyl, such as, for example, methylsulfonyl, or C₁-C₄-haloalkylsulfonyloxy, such as, for example, trifluoromethylsulfonyloxy, also form part of the subject matter of the present invention.

The particularly preferred embodiments of the compounds of the formula XIII with respect to the variables correspond to those of the radicals R¹, R², R³, R⁴, R⁵ and R⁶ of the formula I.

Likewise, particular preference is given to the compounds of the formula XIII, in which L² is halogen, such as, for example, fluorine or chlorine.

Preference is given to compounds of the formula XIII, in which

R¹, R³ are hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl;

R² is C₁-C₆-alkyl;

particularly preferably C₁-C₄-alkyl, such as methyl, ethyl or isopropyl;

especially methyl or ethyl; and

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl or C₁-C₆-haloalkylsulfonyl;

particularly preferably hydrogen, halogen, C₁-C₆-haloalkyl or C₁-C₆-haloalkoxy;

with particular preference hydrogen, fluorine, chlorine, trifluoromethyl or difluoromethoxy;

where R² is not methyl if R¹ and R³ are hydrogen.

Particularly preferred are compounds of the formula XIII in which

R¹ is hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl;

R² is C₁-C₆-alkyl;

particularly preferably C₁-C₄-alkyl, such as methyl, ethyl or isopropyl;

with particular preference methyl or ethyl;

R³ is halogen, cyano, C₁-C₆-alkyl or C₁-C₆-haloalkyl;

particularly preferably halogen, such as fluorine, chlorine or bromine, C₁-C₆-alkyl, such as methyl or ethyl;

with particular preference fluorine, chlorine or methyl.

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulphonyl or C₁-C₆-haloalkylsulphonyl;

particularly preferably hydrogen, halogen, C₁-C₆-haloalkyl or C₁-C₆-haloalkoxy;

with particular preference hydrogen, fluorine, chlorine, trifluoromethyl or difluoromethoxy.

### PREPARATION EXAMPLES

According to process E

2,3,5-Trifluoro-4-methyl-6-(5-trifluoromethyl-3-thienyloxy)-pyridine

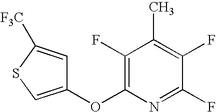

3 g (18 mmol) of 2,3,5,6-tetrafluoro-4-methylpyridine, 3.05 g (18 mmol) of 3-hydroxy-5-trifluoromethylthiophene and 5.02 g (36 mmol) of potassium carbonate in 30 ml of DMF were stirred at room temperature for 48 h. The mixture was diluted with 200 ml of water and then extracted with diethyl ether. The organic phase was washed and dried over sodium sulfate and the solvent was removed. This gave 4.77 g (15.2 mmol, 84%) of the title compound.

¹H-NMR (400 MHz, CDCl₃): δ=2.4 (s, 3H), 7.3 (S, 1H), 7.4 (s, 1H)

3,5-Difluoro-4-methyl-2-(3-trifluoromethyl-1H-pyrazol-1-yl)-6-(5-trifluoromethyl-3-thienyloxy)pyridine

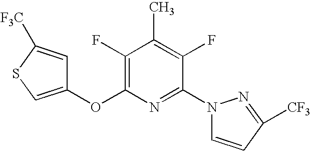

A mixture of 0.2 g (0.64 mmol) of 2,3,5-trifluoro-4-methyl-6-(5-trifluoromethyl-3-thienyloxy)pyridine, 0.08 g (0.59 mmol) of 3-trifluoromethyl-1H-pyrazole and 0.13 g (0.96 mmol) of potassium carbonate in 20 ml of N,N-dimethylformamide was heated at 80° C. for 12 h. The mixture was then diluted with water and ethyl acetate. The aqueous phase was extracted with ethyl acetate, the combined organic phases were washed and dried and the solvent was removed. Column chromatography (petroleum ether/MTBE 8:1→3:1) gave 0.15 g (0.35 mmol, 59%) of the title compound.

¹H-NMR (400 MHz, CDCl₃): δ=2.4 (s, 3H), 6.7 (s, 1H), 7.3 (s, 1H), 7.4 (s, 1H), 8.0 (s, 1H)

According to process A

2-Bromo-4-ethyl-6-(3-trifluoromethyl-1H-pyrazolyl)pyridine

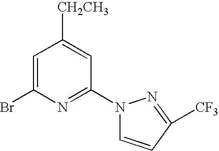

A mixture of 0.95 g (4.7 mmol.) of 2,6-dibromo-4-ethylpyridine, 0.57 g (4.2 mmol) of 3-trifluoromethylpyrazole, 1.21 g (9.4 mmol) of potassium carbonate and 0.13 g (0.9 mmol) of copper(I) bromide in N,N-dimethylformamide (DMF) was stirred at 80° C. for 8 h and then at room temperature for 12 h. The mixture was diluted with water and then extracted with ethyl acetate. The combined organic phases were washed and dried and the solvent was removed. Column chromatography (petroleum ether/ethyl acetate 100:0->8:2) gave 0.43 g (1.3 mmol, 32%) of the title compound.

4-Ethyl-2-(3-trifluoromethyl-1H-pyrazol-1-yl)-6-(5-trifluoro-methyl-3-thienyloxy)pyridine

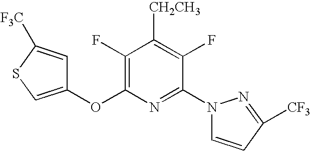

A mixture of 126 mg (0.75 mmol) of 3-hydroxy-5-trifluoro-methylthiophene and 23 mg (0.9 mmol) of sodium hydride in DMF was stirred at 30° C. for 30 min. At 120° C., 200 mg (0.62 mmol) of 2-bromo-4-ethyl-6-(3-trifluoromethyl-1H-pyrazolyl)pyridine in DMF and a spatula tip of copper(I) bromide were then added. The mixture was stirred at 120° C. for 16 h, water was then added and the mixture was extracted with ethyl acetate. The combined organic phases were washed and dried and the solvent was removed. Column chromatography (petroleum ether/ethyl acetate 100:0->0:100) gave 120 mg (0.29 mmol, 48%) of the title compound.

In addition to the above compounds, Tables 2 and 3 list further 4-alkyl-substituted thienyloxypyridines of the formula I and thienyloxypyridines of the formula XIII which were prepared or are preparable in an analogous manner by the processes described above.

In addition to the above compound, Table 4 lists further 3-trifluoromethyl-1H-pyrazol-1-yl-substituted pyridines of the formula III which are prepared in an analogous manner by the process described below.

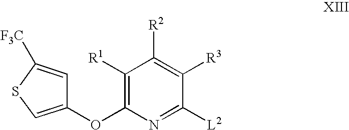

(where R⁴=5-CF₃, R⁵═H, R⁶═the thienyl radical is attached in the 3-position)

TABLE 2

| No. | R¹ | R² | R³ | L² | ¹H-NMR[400 MHz, CDCl₃] |
|---|---|---|---|---|---|
| 2.1 | F | CH₃ | F | F | 2.4(s, 3H), 7.3(s, 1H), 7.4(s, |
| | | | | | 1H) |

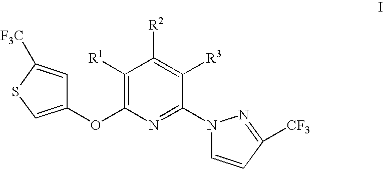

(where R⁴=5-CF₃, R⁵H, R⁶═H;

the thienyl radical is attached in the 3-Position)

TABLE 3

| No. | R¹ | R² | R³ | ¹H-NMR(400 MHz, CDCl₃) |
|---|---|---|---|---|
| 3.1 | F | CH₃ | F | 2.4(s, 3H), 6.7(s, 1H), 7.3(s, |
| | | | | 1H), 7.4(s, 1H), 8.0(s, 1H) |
| 3.2 | Br | CH₃ | Br | 2.7(s, 3H), 6.6(s, 1H), 7.3(s, |
| | | | | 1H), 7.4(s, 1H), 7.8(s, 1H) |
| 3.3 | H | CH₂CH₃ | H | 1.3(t, 3H), 2.8(q, 2H), 6.6(s, |
| | | | | 1H), 6.8(s, 1H), 7.2(s, |
| | | | | 1H), 7.4(s, 1H), 7.6(s, 1H), |
| | | | | 8.2(s, 1H) |

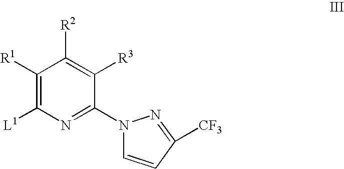 TABLE 4

| No. | R¹ | R² | R³ | L¹ | ¹H-NMR[400 MHz, CDCl₃] |
|---|---|---|---|---|---|
| 4.1 | Br | CH₃ | Br | Br | 2.8(s, 3H), 6.8(s, 1H), 8.0(s, |
| | | | | | 1H) |
| 4.2 | H | CH₂CH₃ | H | Br | 1.2(t, 3H), 2.7(q, 2H), 6.7(2, |
| | | | | | 1H), 7.3(s, 1H) 8.8(s, 1H), 8.5(s, |
| | | | | | 1H) |

Use

The 4-alkyl-substituted thienyloxypyridines of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and harmful grasses in crops such as wheat, rice, maize, soya and cotton without causing any significant damage to the crop plants. This effect is mainly observed at low rates of application.

Depending on the application method used, the compounds of the formula I or the herbicidal compositions comprising them can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia., Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

The compounds of the formula I, or the herbicidal compositions comprising them, can be used for example in the form of ready-to-spray aqueous solutions, powders, suspensions, also highly-concentrated aqueous, oily or other suspensions or dispersions, emulsions, oil dispersions, pastes, dusts, materials for broadcasting or granules, by means of spraying, atomizing, dusting, broadcasting or watering. The use forms depend on the intended aims; in any case, they should ensure a very fine distribution of the active compounds according to the invention.

The herbicidal compositions comprise a herbicidally effective amount of at least one compound of the formula I or an agriculturally useful salt of I and auxiliaries customary for formulating crop protection agents.

Essentially, suitable inert auxiliaries include:

mineral oil fractions of medium to high boiling point, such as kerosene and diesel oil, furthermore coal tar oils and oils of vegetable or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates consisting of active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl polyglycol ether or tributylphenyl polyglycol ether, alkylaryl polyether alcohols, isotridecyl alcohol, fatty alcohol ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials., fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise from about 0.001 to 98% by weight, preferably 0.01 to 95% by weight of at least one active compound. The active compounds are employed in a purity of from 90% to 100%, preferably from 95% to 100% (according to the NMR spectrum).

The production of such preparations is illustrated by the following formulation examples:

I. 20 parts by weight of an active compound of the formula I are dissolved in a mixture consisting of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II. 20 parts by weight of an active compound of the formula I are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III. 20 parts by weight of an active compound of the formula I are dissolved in a mixture consisting of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous-dispersion which comprises 0.02% by weight of the active compound.

IV. 20 parts by weight of an active compound of the formula I are mixed thoroughly with 3 parts by weight of sodium diisobutylnaphthalenesulfonate, 17 parts by weight of the sodium salt of lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V. 3 parts by weight of an active compound of the formula I are mixed with 97 parts by weight of finely divided kaolin. This gives a dust which comprises 3% by weight of the active compound.

VI. 20 parts by weight of an active compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII. 1 part by weight of an active compound of the formula I is dissolved in a mixture consisting of 70 parts by weight of cyclohexanone, 20 parts by weight of ethoxylated isooctylphenol and 10 parts by weight of ethoxylated castor oil. This gives a stable emulsion concentrate.

VIII. 1 part by weight of an active compound of the formula I is dissolved in a mixture of 80 parts by weight of cyclohexanone and 20 parts by weight of Wettol^{R} EM 31

(=nonionic emulsifier based on ethoxylated castor oil). This gives a stable emulsion concentrate.

The compounds of the formula I or the herbicidal compositions can be applied pre- or post-emergence. If the active compounds are less well tolerated by certain crop plants, application techniques may be used in which the herbicidal compositions are sprayed, with the aid of the spraying equipment, in such a way that they come into contact as little as possible, if at all, with the leaves of the sensitive crop plants, while the active compo unds reach the leaves of undesirable plants growing underneath, or the bare soil surface (post-directed, lay-by).

The application rates of the compound of the formula I are from 0.001 to 3.0, preferably from 0.01 to 1.0 kg/ha of active substance (a.s.), depending on the control target, the season, the target plants and the growth stage.

To widen the activity spectrum and to achieve synergistic effects, the 4-alkyl-substituted thienyloxypyridines of the formula I may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (hetero)aryloxyalkanoic acids and their derivatives, benzoic acid and its derivatives, benzothiadiazinones, 2-(hetaroyl/aroyl)-1,3-cyclohexanediones, heteroarylaryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ether, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds of the formula I, alone or else concomitantly in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

### USE EXAMPLES

The herbicidal activity of the 4-alkyl-substituted thienyloxypyridines of the formula I was demonstrated by the following greenhouse experiments:

The cultivation containers used were plastic flowerpots containing loamy sand with approximately 3.0% of humus as the substrate. The seeds of the test plants were sown separately for each species.

For the pre-emergence treatment, directly after sowing the active compounds, which had been suspended or emulsified in water, were applied by means of finely distributing nozzles. The containers were irrigated gently to promote germination and growth and subsequently covered with transparent plastic hoods until the plants had rooted. This cover caused uniform germination of the test plants, unless this was adversely affected by the active compounds.

For the post-emergence treatment, the test plants were first grown to a height of from 3 to 15 cm, depending on the plant habit, and only then treated with the active compounds which had been suspended or emulsified in water. The test plants were for this purpose either sown directly and grown in the same containers, or they were first grown separately as seedlings and transplanted into the test containers a few days prior to treatment. The application rate for the post-emergence treatment was 0.25 or 0.125 kg of a.s. (active substance)/ha.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the above-ground parts, and 0 means no damage, or normal course of growth.

The plants used in the greenhouse experiments were of the following species:

| Scientific name | Common name |
|---|---|
| Amaranthus retroflexus | pig weed |
| Chenopodium album | lamb's quarters |
| Galium aparine | catchweed |
| Pharbitis purpurea | tall morningglory |

At application rates of 0.25 or 0.125 kg/ha, the compound 3.1 (Table 3) showed very good post-emergence action against the undesirable plants Amaranthus retroflexus, Chenopodium album, Galium aparine and Pharbitis purpurea.

1. A 4-alkyl-substituted thienyloxypyridine of the formula I 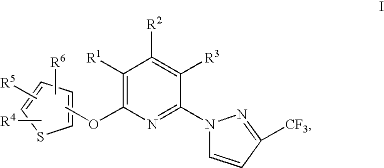
where
R¹, R³ are hydrogen, halogen, cyano, nitro, C₁-C₆-alkyl, C₁-C₆-haloalkyl, C₁-C₆-alkoxy or C₁-C₆-haloalkoxy;
R² is C₁-C₆-alkyl or C₃-C₆-cycloalkyl;
R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio,
C₁-C₆-alkylsulfonyl or C₁-C₆-haloalkylsulfonyl;
where R² is not methyl if R¹ and R³ are hydrogen;
and its agriculturally useful salts.

2. A 4-alkyl-substituted thienyloxypyridine of the formula I as claimed in claim 1 where
R² is methyl;
where R¹ and R³ are not simultaneously hydrogen.

3. A 4-alkyl-substituted thienyloxypyridine of the formula I as claimed in claim 1
where
R² is C₁-C₆-alkyl.

4. A 4-alkyl-substituted thienyloxypyridine of the formula I as claimed in claim 1 where
R² is C₃-C₆-cycloalkyl.

5. A 4-alkyl-substituted thienyloxypyridine of the formula I as claimed in claim 1 where
R⁴, R⁵, R⁶ are hydrogen, halogen, cyano, C₁-C₆-alkyl, C₁-C₆-haloalkyl or C₁-C₆-haloalkoxy;

6. A process for preparing a 4-alkyl-substituted thienyloxypyridine of the formula I as claimed in claim 1, which comprises reacting 3-trifluoromethyl-1H-pyrazol-1-yl-substituted pyridines of the formula III 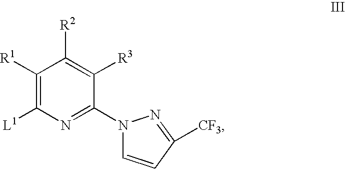
where R¹, R² and R³ are as defined in claim 1 and L¹ is a nucleophilically displaceable leaving group,
with a hydroxythiophene of the formula II 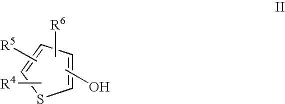
where R⁴, R⁵ and R⁶ are as defined in claim 1.

7. A process for preparing a 4-alkyl-substituted thienyloxypyridines of the formula I as claimed in claim 1, which comprises reacting thienyloxypyridine derivatives of the formula XIII 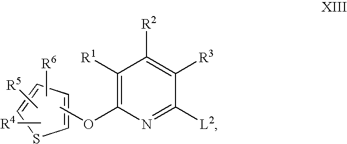
where R₁, R², R³, R⁴, R⁵ and R⁶ are as defined in claim 1 and L² is a nucleophilically displaceable leaving group,
with a pyrazole derivative of the formula IV

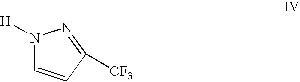

8. A 3-trifluoromethyl-1H-pyrazol-1-yl-substituted pyridine derivative of the formula III 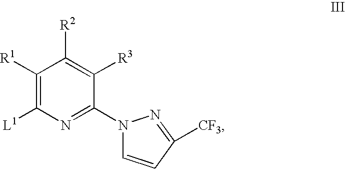
where R¹, R² and R³ are as defined in claim 1 and L¹ is a nucleophilically displaceable leaving group.

9. A thienyloxypyridine derivative of the formula XIII 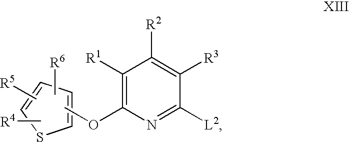
where R¹, R², R³, R⁴, R⁵ and R⁶ are as defined in claim 1 and L² is halogen, C₁-C₄-alkylsulfonyl, C₁-C₄-alkylsulfonyloxy, C₁-C₄-haloalkylsulfonyloxy or trialkylammonium.

10. A composition, comprising a herbicidally effective amount of at least one 4-alkyl-substituted thienyloxypyridine of the formula I or of an agriculturally useful salt of I as claimed in claim 1 and auxiliaries customary for formulating crop protection agents.

11. A process for preparing compositions as claimed in claim 10, which comprises mixing a herbicidally effective amount of the at least one 4-alkyl-substituted thienyloxypyridine derivative of the formula I or of an the agriculturally useful salt of I and auxiliaries customary for formulating crop protection agents.

12. A method for controlling undesirable vegetation, which comprises allowing a herbicidally effective amount of at least one 4-alkyl-substituted thienyloxypyridine derivative of the formula I or of an agriculturally useful salt of I as claimed in claim 1 to act on plants, their habitat and/or on seeds.

13 (canceled)
